(19) United States
(12) Patent Application Publication
(10) Pub. No.: US 2025/0072766 A1
(43) Pub. Date: Mar. 6, 2025

(54) WEARABLE DEVICE FOR MONITORING BODY TEMPERATURE

(71) Applicant: Diane Alexander, Stone Mountain, GA (US)

(72) Inventor: Diane Alexander, Stone Mountain, GA (US)

(21) Appl. No.: 18/819,039

(22) Filed: Aug. 29, 2024

Related U.S. Application Data
(60) Provisional application No. 63/579,721, filed on Aug. 30, 2023.

(51) Int. Cl. A61B 5/01; A61B 5/00

(52) U.S. Cl. CPC A61B 5/01; A61B 5/6826

(57) ABSTRACT

A wearable device for monitoring body temperature that can be worn on a finger as a finger ring. The wearable device can include a temperature sensor that can measure body temperature continuously; and display the measured body temperature on a display of the wearable device.

### CROSS-REFERENCE TO RELATED APPLICATIONS

This application claims priority from a U.S. provisional patent application Ser. No. 63/579,721, filed on Aug. 30, 2023, which is incorporated herein by reference in its entirety.

### FIELD OF INVENTION

The present invention relates to a device for monitoring body temperature, and more particularly, the present invention relates to a wearable device for continuous monitoring of body temperature.

### BACKGROUND

In healthy individuals, body temperature is regulated by the hypothalamus to remain within a normal temperature range. In normal body conditions, bodily functions can be adjusted according to the external environment so that the body temperature remains within the normal temperature range. However, certain external factors can change the normal body temperature, such as infections. Thus, a change in body temperature from normal body temperature is a primary symptom of abnormality in the human body. A body temperature higher than normal body temperature is generally the first symptom of microbial infection. The change in body temperature may also be a result of some internal body disorder. However, it is well established that a change in body temperature from the normal temperature range is a primary symptom of abnormality.

Body temperature monitoring is also used in public places to detect people who may be suffering from a contagious infection. For example, during the COVID-19 pandemic, screening of individuals based on external body temperature was used to identify individuals that have fever, which may be due to COVID-19 infection. Similarly, screening based on body temperature is used to identify infected people during epidemics.

Thus, a device that can detect changes in body temperature can help diagnose a disease at an early stage. Early diagnosis can help prevent the spread of infectious diseases. Also, an early treatment can be started to control the progression of the disease.

Thus, there is a need for a device that can monitor body temperature continuously.

The term “continuous” herein refers to continuous intervals, for example. Every hour, every 8 hours, and the like intervals.

### SUMMARY OF THE INVENTION

The following presents a simplified summary of one or more embodiments of the present invention to provide a basic understanding of such embodiments. This summary is not an extensive overview of all contemplated embodiments and is intended to neither identify critical elements of all embodiments nor delineate the scope of any or all embodiments. Its sole purpose is to present some concepts of one or more embodiments in a simplified form as a prelude to the more detailed description that is presented later.

The principal object of the present invention is therefore directed to a wearable device that can continuously monitor body temperature.

Another object of the present invention is that the wearable device is economical to manufacture.

Still, another object of the present invention is that the wearable device can be worn on a finger.

Yet another object of the present invention is that the wearable device has an aesthetic appearance.

A further object of the present invention is that the wearable device is comfortable to wear.

Yet an object of the present invention is that the wearable device can be weatherproof.

An additional object of the present invention is that the wearable device can display real-time notifications.

In one aspect, disclosed is a wearable device for monitoring body temperature, the wearable device comprises a housing adapted as a ring configured to be worn on a finger of a user; and a heat sensor configured to detect body temperature from the finger, the heat sensor encased within the housing. The wearable device further comprises a control unit. The control unit is configured to receive a measured temperature value from the heat sensor, compare the measured temperature value against a predefined temperature range, and display to the user when the measured temperature value is outside the predefined temperature range.

In one aspect, disclosed is a method for monitoring a body temperature of a user, the method comprising adapting a housing in a form of a finger ring, encasing a heat sensor within the housing, the heat sensor is configured to detect the body temperature, coupling a control unit with the heat sensor to form a wearable device, the control unit encased within the housing. The method further comprises wearing the wearable device on a finger by the user. The method further comprises receiving, by the control unit, a measured temperature value from the heat sensor; comparing, by the control unit, the measured temperature value against a predefined temperature range; and displaying for the user on the face of the ring, when the measured temperature value is outside the predefined temperature range.

### BRIEF DESCRIPTION OF DRAWINGS

The accompanying figures, which are incorporated herein, form part of the specification and illustrate embodiments of the present invention. Together with the description, the figures further explain the principles of the present invention and to enable a person skilled in the relevant arts to make and use the invention.

FIG. 1 is a perspective view of a wearable device, according to an exemplary embodiment of the present invention.

FIG. 2 is a block diagram showing the architecture of the wearable device, according to an exemplary embodiment of the present invention.

### DETAILED DESCRIPTION

Subject matter will now be described more fully hereinafter with reference to the accompanying drawings, which form a part hereof, and which show, by way of illustration, specific exemplary embodiments. Subject matter may, however, be embodied in a variety of different forms and, therefore, covered or claimed subject matter is intended to be construed as not being limited to any exemplary embodiments set forth herein; exemplary embodiments are provided merely to be illustrative. Likewise, a reasonably broad scope for claimed or covered subject matter is intended. Among other things, for example, the subject matter may be embodied as methods, devices, components, or systems. The following detailed description is, therefore, not intended to be taken in a limiting sense.

The word “exemplary” is used herein to mean “serving as an example, instance, or illustration.” Any embodiment described herein as “exemplary” is not necessarily to be construed as preferred or advantageous over other embodiments. Likewise, the term “embodiments of the present invention” does not require that all embodiments of the invention include the discussed feature, advantage, or mode of operation.

The terminology used herein is to describe particular embodiments only and is not intended to be limiting to embodiments of the invention. As used herein, the singular forms “a”, “an” and “the” are intended to include the plural forms as well, unless the context clearly indicates otherwise. It will be further understood that the terms “comprises”, “comprising,”, “includes” and/or “including”, when used herein, specify the presence of stated features, integers, steps, operations, elements, and/or components, but do not preclude the presence or addition of one or more other features, integers, steps, operations, elements, components, and/or groups thereof.

The following detailed description includes the best currently contemplated mode or modes of carrying out exemplary embodiments of the invention. The description is not to be taken in a limiting sense but is made merely to illustrate the general principles of the invention since the scope of the invention will be best defined by the allowed claims of any resulting patent.

Disclosed is a wearable device that can be used to monitor body temperature. The disclosed device can be worn on a finger and can detect the body temperature. The disclosed device can continuously monitor the body temperature from the finger.

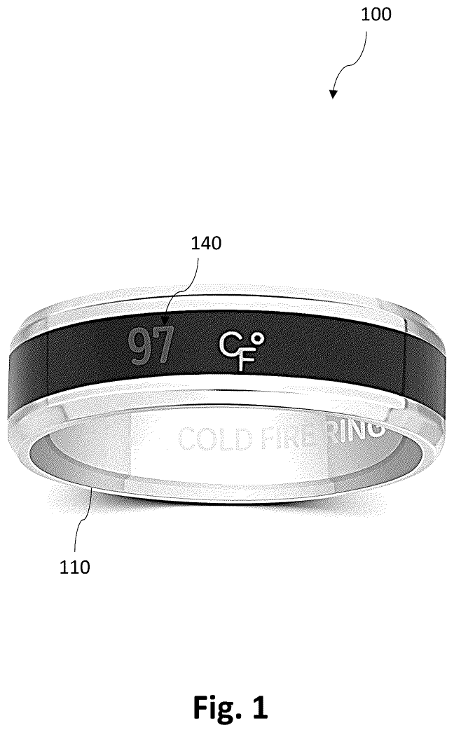

The disclosed wearable device can be manufactured in the form of a ring. FIG. 1 shows an example of the wearable device 100 in the form of a ring. The wearable device includes a housing 110 that can be adapted in the form of a finger ring. The housing can encase the display of the disclosed wearable device. The housing can be made in the form of a ring that can be worn on a finger. The housing can be made in different sizes to fit different sizes of fingers. Display 160 can also be seen in FIG. 1 in which the information can be displayed.

The housing can be made from lustrous heat conducting titanium steel which provides an aesthetic appearance. The housing is made of hypoallergenic heat conducting titanium steel

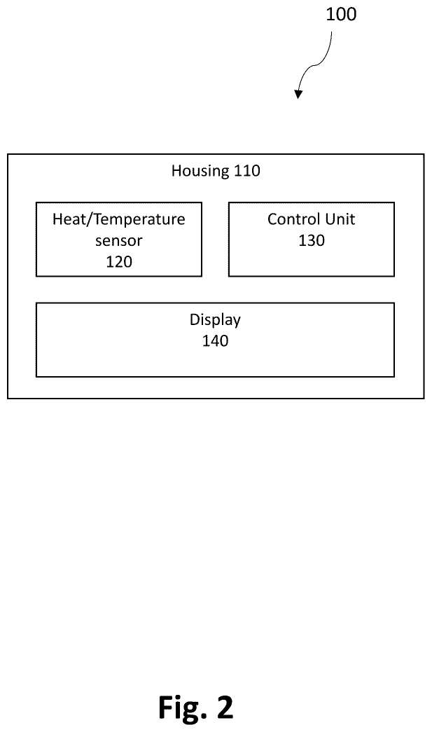

Referring to FIG. 2 which is a block diagram showing the architecture of the disclosed wearable device. The wearable device 100 includes a suitable heat sensor 120, such as a thermometer for measuring the body temperature. The housing made of heat conducting titanium steel conducts body heat emitted from the finger to the housing surface incorporating the heat/temperature sensor 120.

The control unit 130 upon detecting measured body temperature can display the measured temperature on the display 140. The display can present measured temperature values in numerical form. The visual indicator is in the form of a digital display.

In certain implementation, disclosed is a method of identifying individuals and groups of individuals using the disclosed device in early stages of disease course by way of temperature elevation monitoring. For example, there is a time gap between the onset of the elevation in temperate and the person realizing the high temperature. This time gap can be about 4-6 hours; however, the time gap, also referred to as time gap period, can vary without departing from the scope of the present invention. Time gap is a duration of time when individuals may show an elevation in temperature prior to when they actually know they are sick. Monitoring the temperature of individuals and individuals within groups simultaneously by use of disclosed wearable device, allows for preemptive measures to be taken to mitigate unimpeded contagious transmission of germs thus slowing the rapid spread of infection.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above-described embodiment, method, and examples, but by all embodiments and methods within the scope and spirit of the invention as claimed.

What is claimed is:

1. A wearable device for monitoring body temperature, the wearable device comprises:
a housing adapted as a ring configured to be worn on a finger of a user; and
a heat sensor configured to measure body temperature from the finger, the heat sensor encased within the housing.

2. The wearable device according to claim 1, wherein the wearable device further comprises:
a display disposed on an outer surface of the housing; and
a control unit configured to display the measured body temperature on the display.

3. The wearable device according to claim 2, wherein the housing is made of sensitive thermal conducting titanium steel which transfers heat from the finger to the heat sensor.

4. A method for monitoring a body temperature of a user, the method comprising:
adapting a housing in a form of a finger ring;
encasing a heat sensor within the housing, the heat sensor is configured to measure the body temperature from a finger in which the device is worn;
coupling a control unit with the heat sensor, the control unit encased within the housing;
coupling a display to an external surface of the housing; and
displaying the measured temperature on the display.

5. The method according to claim 4, wherein the method further comprises:
wearing the wearable device on the finger by the user.

6. The method according to claim 5, wherein the housing is made of sensitive thermal conducting titanium steel which transfers heat from the finger to the heat sensor.
